(19) United States
(12) Patent Application Publication
(10) Pub. No.: US 2005/0015207 A1
(43) Pub. Date: Jan. 20, 2005

(54) Method and apparatus for object based biological information, manipulation and management

(76) Inventor: Burra V.L.S. Prasad, Hyderabad (IN)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/873,923

(22) Filed: Jun. 21, 2004

Related U.S. Application Data
(60) Provisional application No. 60/480,618, filed on Jun. 20, 2003.

(51) Int. Cl. G06F 19/00; G01N 33/48; G01N 33/50

(52) U.S. Cl. 702/19

(57) ABSTRACT

A biological data manipulation system, and a programming language and system, and a method of use thereof, are disclosed. The system, apparatus, and method include a first data file receiver for receiving a first data file having data indicative of a first data file type and data indicative of at least one biological data object, a first classifier that applies a plurality of rules to the first data file to parse the first data file into a first data file type and into a plurality of string classes, a second classifier that differentiates a master class for ones of the plurality of string classes, wherein the master class is differentiated against at least one selected from the group consisting of a single biosequence master and a multiple biosequence master, and a third classifier that classifies an at least one biological data object of the first data file, wherein the at least one biological data object is multiple inherited to the master class in accordance with at least one of the plurality of rules, and in accordance with at least a partial sequence of stored biodata compared by the third classifier against at least a partial sequence of at least one of the plurality of string classes.

### CROSS-REFERENCE TO A RELATED APPLICATION

This application claims the benefit of U.S. provisional application Ser. No. 60/480,618, filed Jun. 20, 2003.

### FIELD OF THE INVENTION

The present invention is directed generally to a method and apparatus for manipulating information and managing information between points and, more particularly, to an apparatus and method for object based biological information manipulation and management.

### BACKGROUND OF THE INVENTION

Researchers utilizing computers to enhance research capabilities often face the difficult task of programming in computer languages and software programs not designed for scientific applications. Trying to compile results from a variety of off-the-shelf programs into a single unified and useable database can be an extremely difficult task. Further, compiling the numerous and varied data stores and databases applicable to biological research, including structural databases, sequence databases, genomic databases, metabolism databases, and similar databases, for accessing by a single program or related programming set, is very difficult.

Thus, an obstacle for a biological researcher is the time spent writing code for parsing file formats of data retrieved from these existing and varied databases with the goal of analyzing the retrieved data in a unified system. This time spent by the researcher is non-productive, and time spent on valuable research activities could be increased if the researcher was provided with more efficient tools to access and manipulate this desired information.

Several generations of biological programming have yet to solve many of the difficulties faced by researchers dependent on computerization. A first generation of biosoftware was not object oriented (“OO”), and hence included small, isolated, stand alone applications having specific, pre-determined objectives. This first generation of software included programs designed for structure alignment (such as ALIGN), structure validation (PROCHECK and WHATIF), database searching for sequence homologies (BLAST: FASTA), pair wise and multiple sequence alignment (CLUSTALW), surface area calculations and shape complementarity (MSP, NACCESS), multiple structure alignment (STAMP), and for visualization of macromolecules (RASMOL, FRODO, and MOLSCRIPT). These programs are highly limited in scope and make it necessary for the researcher to utilize many different programs to manipulate one piece of data multiple ways.

Second generation biosoftware has abstracted to improve user convenience as part of the objective. Second generation programs include a collection and compilation of a large set of disparate programs compiled together, wherein each individual program is similar to first generation software. Programs such as GCG and CCP4 suite belong to this second generation. Although these collections of individual programs can organize and compile information together into a single package, the programs are independent executables and cannot communicate nor collaborate with one other. The use of scripting languages can allow for communication and collaboration between programs, but at a tremendous cost of efficiency and speed.

Second generation biosoftware, like first generation software, does not support OO programming. A programmer has to follow strict syntactic and semantic rules which can differ between software packages, thereby making jumps between software packages difficult. Additionally, the code produced from these procedural packages is far from simple or efficient. These programs do not automatically scale up, and are inflexible closed systems. Thus, the first and second generation biosoftware could not appropriately handle the ever-expanding library of biological terms and processes.

With the advent of whole genome projects, the amount of data to be analyzed and/or simulated is many orders of magnitude higher than in the very recent past. The need to handle large scale data analysis and simulation has created a third generation of biosoftware based on the OO platform. This third generation has been created to overcome the drawbacks of procedural languages. The starting point is to build a user's model by creating “objects.” These objects are “data structures” encapsulated with a set of routines called “methods”, which methods operate on the data. Objects can also have “attributes.” An example would be the attribute employee number (5 digits) of an employee object. An access method could be “get employee.” Operations on the data can only be performed via these methods.

Objects having similar behavior can be grouped in the same “class.” Classes are arranged in a class “hierarchy”. Classes and subclasses let objects in a subclass “inherit” everything from the respective super class. In an OO development application, objects use the services of other objects, which in turn use the service of other objects, and so on. Several attempts have been made at creating biosoftware in an OO platform, most having abstracted only the sequence domain. This leaves the utility of the bio-OO platform restricted to sequence analysis. Other bio-OO efforts have very limited and specific stand alone libraries.

Therefore, the need exists to provide the user with a method and apparatus for an object based biological programming environment that includes a hierarchical organization for biodata, that encourages creativity, that enables the researcher to quickly test and compare multiple alternatives, that allows for the re-use of data and the expansion of data libraries, that entails the abstraction needed to efficiently handle complex biological data, and that provides for the inclusion of databases operating on mis-matched protocols.

### BRIEF SUMMARY OF THE INVENTION

The present invention includes a programming language, system, and tool for a biologist to develop, manipulate, and manage biological data using an object-oriented paradigm (OOP), supported by programming languages such as C++. The present invention may provide a set of Biological Abstract Data Types (BioADTs) that a programmer can simplistically use to program in biological terminology. An ADT defines a concept independent of programming language. A representation of an ADT in OOP is herein called Class. The present invention uses a class and inheritance OOP system to provide an extensible, maintainable, reusable and biologist friendly bio-programming environment that encourages creativity in exploratory research and flexibility in developing bio-computational applications.

The present invention may include a biological data manipulation system, and a programming language and system, including a first data file receiver for receiving a first data file having data indicative of a first data file type and data indicative of at least one biological data object, a first classifier that applies a plurality of rules to the first data file to parse the first data file into a first data file type and into a plurality of string classes (e.g., nucleic acids, coordinates of atoms and 3D structure of proteins, and/or other data suitable for placement or storage in one or more string classes), a second classifier that differentiates a master class for ones of the plurality of string classes, wherein the master class is differentiated against at least one selected from the group consisting of a single biosequence master and a multiple biosequence master, and a third classifier that classifies an at least one biological data object of the first data file, wherein the at least one biological data object is multiple inherited to the master class in accordance with at least one of the plurality of rules, and in accordance with at least a partial sequence of stored biodata compared by the third classifier against at least a partial sequence of at least one of the plurality of string classes.

Thus, the present invention provides the user with a method and apparatus for an object based biological programming environment which includes a hierarchical organization for biodata, that encourages creativity, that enables the researcher to quickly test and compare multiple alternatives, that allows for the re-use of data and the expansion of data libraries, that entails the abstraction needed to efficiently handle complex biological data, and that provides for the inclusion of databases operating on mis-matched protocols.

Preferably and according to an additional and optional aspect, the invention also provides for an internal interpreter means, which is capable of processing biological programming language features. Such interpreter means enable the user to have a programming environment feature, thereby having the advantage of avoiding compilation and linking of the code. Such an interpreter will enable the processing of language features, using the set of defined classifiers according to the present invention. This optional features can be applied to the biological feature manipulation system, the method and/or the computer-readable medium, carrying respective data and information according to the present invention.

The present invention thereby succeeds in providing a very effective biological programming environment and discovery system and therefore providing a very useful and effective tool for a biologist.

Those and other advantages and benefits of the present invention will become apparent from the detailed description of the invention hereinbelow.

### BRIEF DESCRIPTION OF THE SEVERAL VIEWS OF THE DRAWING

For the present invention to be clearly understood and readily practiced, the present invention will be described in conjunction with the following figures, wherein like reference numerals designate like elements, and wherein:

FIG. 1 is a block diagram illustrating an embodiment of the structure of the present invention;

FIG. 1A is a block diagram illustrating a embodiment of the system of the present invention;

FIG. 2 is a block diagram illustrating an embodiment of the multiply derived hierarchy of the present invention;

FIG. 2A is a block diagram illustrating an embodiment of the multiply derived hierarchy of the present invention;

FIG. 2B is a block diagram illustrating an embodiment of the multiply derived hierarchy of the present invention;

FIG. 2C is a block diagram illustrating an embodiment of the multiply derived hierarchy of the present invention;

FIG. 3 is a block diagram illustrating an embodiment of the multiply derived hierarchy of the present invention;

FIG. 4 is a block diagram illustrating a biological data manipulator, a manipulation system, and at least one programming hierarchy and system;

FIG. 5 is a block diagram illustrating at least one classifier of the present invention for use in the system of FIG. 1;

FIG. 5A is a block diagram illustrating at least one sequence format converter of the present invention for use in the system of FIG. 1; and

FIG. 6 is a block diagram illustrating an embodiment of a data library for use in the present invention.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, many other elements found in a typical information management system and method. Those of ordinary skill in the art will recognize that other elements are desirable and/or required in order to implement the present invention. However, because such elements are well known in the art, and because they do not facilitate a better understanding of the present invention, a discussion of such elements is not provided herein.

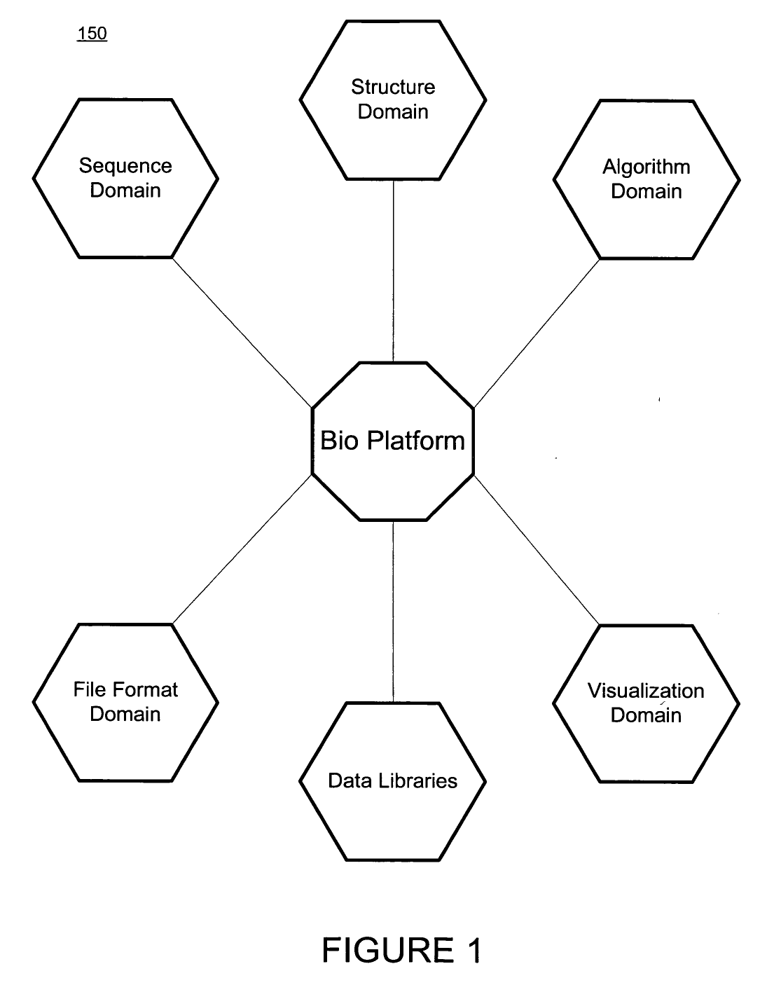

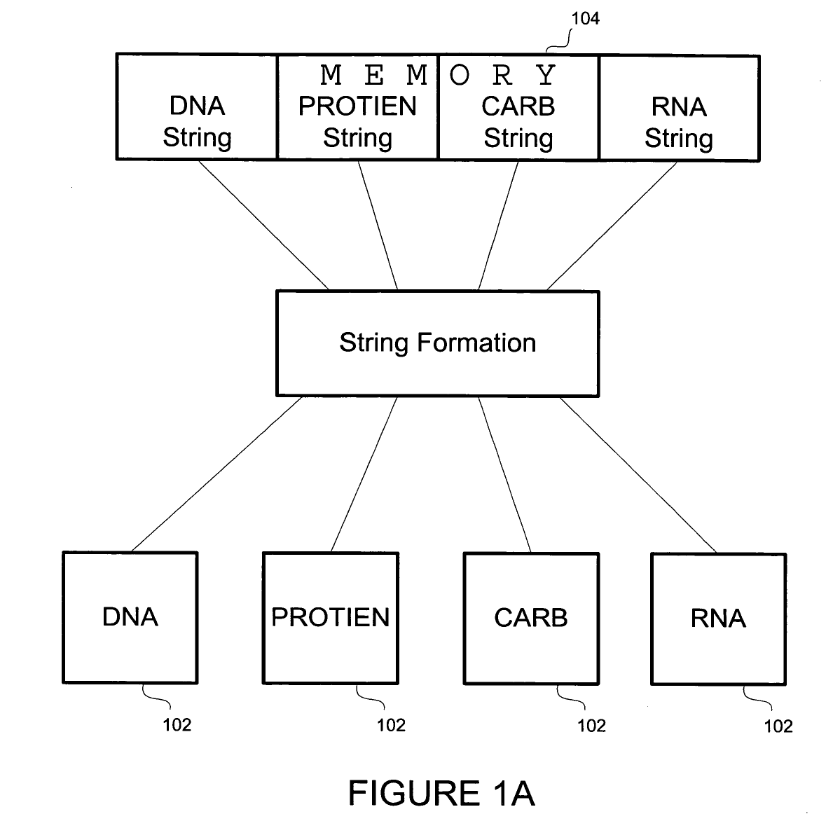

Objected Oriented Paradigm (“OOP”) overcomes many difficulties inherent in other programming paradigms, such as an imperative programming paradigm like Pascal, a logic programming paradigm like Prolog, or a functional programming paradigm like Haskell. OOP can overcome the inherent difficulties of other paradigms by reducing the problem space to deal with increasing complexity. OOP reduces the problem space, and provides scalability, through three properties, namely data abstraction, data encapsulation and inheritance. Data abstraction divides a complex problem into simple and conceptually independent entities that form the building blocks of a project. The abstracted entities then can inter-communicate and collaborate to simulate a complex phenomenon by obeying a defined behavior. An exemplary specific embodiment of the biological abstraction provided in the present invention is illustrated in FIG. 1. The defined behavior or state of the abstracted entities is data encapsulation. Data encapsulation segregates what is done from how something is done, thereby giving the programmer the ability to modify and improve techniques without disturbing underlying data. The reduction of the problem space using these three properties occurs by using the three properties to form a hierarchy of inheritance. Code optimization and code reuse may be employed through the use of a hierarchical ordering.

FIG. 1 is a block diagram illustrating the manner in which such a hierarchy is created in accordance with the present invention. As illustrated, a bio-platform may be provided, wherein the bio-platform accesses objects exterior, or within, or related to the bio-platform. For example, the bio-platform may access master domains, as those domains relate to biological abstraction. In the illustrated example, biological abstraction is performed to abstract biological entities into one or more of the sequence of the biological entity, the structure of the biological entity, and/or the algorithm to be applied to the biological entity. Thus, for example, a DNA sequence might fall within the sequence domain, a molecular structure might fall within the structure domain, and an assessment of molecular weight might fall within the algorithmic domain.

As will be apparent to those skilled in the art in light of the disclosure herein, the abstraction of the biological entities into a master domain allows subsequent abstraction, within the selected domain, into one or more additional levels of abstraction, such as the codons within a DNA sequence or amino acids in a protein sequence. Further, the abstraction, as illustrated, may allow the intercommunication of different domains, and/or of lower hierarchical layers within each domain, such as the application of algorithms within the sequence domain to sequences within the sequence domain.

Further, as shown in FIG. 1, the abstraction of biological entities into the hierarchy of the present invention allows interaction with elements from other domains. For example, a file format domain that allows the bio-platform to assess, or formulate, the file type of a given file, may be provided. Further, data libraries, such as those known in the art as related to biological entities, may be provided, to allow for interoperability with input biological sequences, for example, upon application of one or more bioligical algorithms to the input sequences, for example. Additionally, visualization domains may be provided, such as to provide interfaces to the bio-platform, and each other of the domains of the bio-platform, to a user.

The visualization domain, in a preferred embodiment of the invention, is further abstracted into BioGL class which may be dependent on BioData class. The BioData class may have Bio2Ddata (two variables) and Bio3Ddata (three variables).

For example, using a compiler and an operating system, such as a C++ compiler, v2.95 running in Mandrake Linux, v8.1, a biological data manipulation and management system and language in accordance with the present invention may be implemented. The system and language of the present invention may, in this exemplary embodiment, assist to explain and manipulate biological entities, such as DNA, protein, or carbohydrates, for example. These biological entities have data and have a defined behavior or state associated therewith, making these entities candidates to form BioADTs. For example, a group of BioADTs, having an encapsulation and interface to inter-communicate and collaborate, may provide a class system, such as within the domain hierarchy of FIG. 1, to describe the biological complexity of the biological entities. A set of BioADT classes allow for the simulation of interaction of biological entities, such as the complex molecular interactions within a cell.

These biological entity classes may describe biological sequence information or structure information, for example, as illustrated in FIG. 1. The biological sequence information may be stored as a string datatype and structure information may be stored in user defined structures such as BioPoint and BioAtom, for example, as illustrated in FIG. 1A. For example, any sequence, such as a sequence of genomes, genes, cDNAs, mRNAs, tRNAs, plasmids, ESTs, SNPs or proteins 102, may be stored 104 as a string datatypes, which string is a standard C++ library class. A string class may allow for the performance of pattern searching, matching, counting, comparing, substring fetching, and the like. The string class may then be qualified with a sequence name. For example, a BioSequence class may be implemented from a series of biosequence ADTs to form the base class for derived sequence classes.

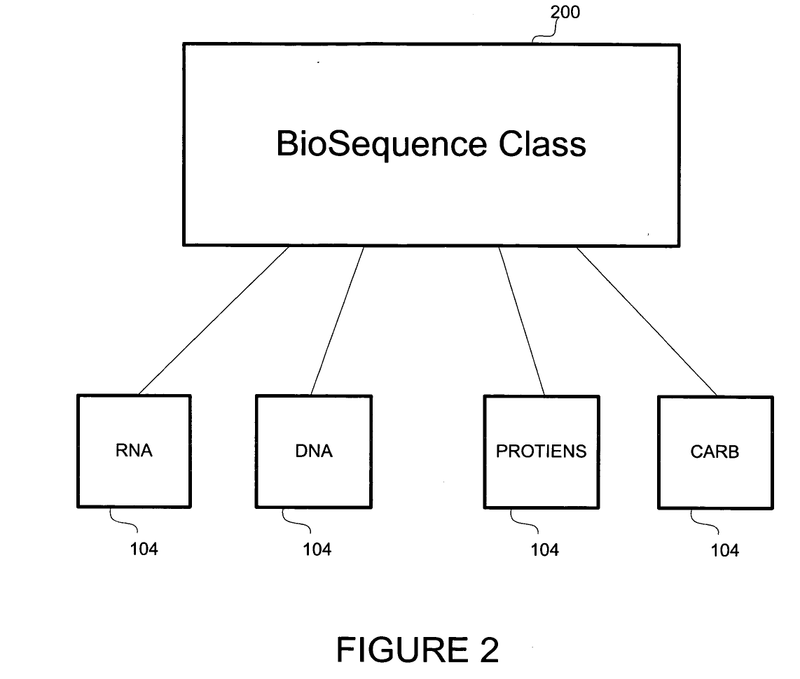

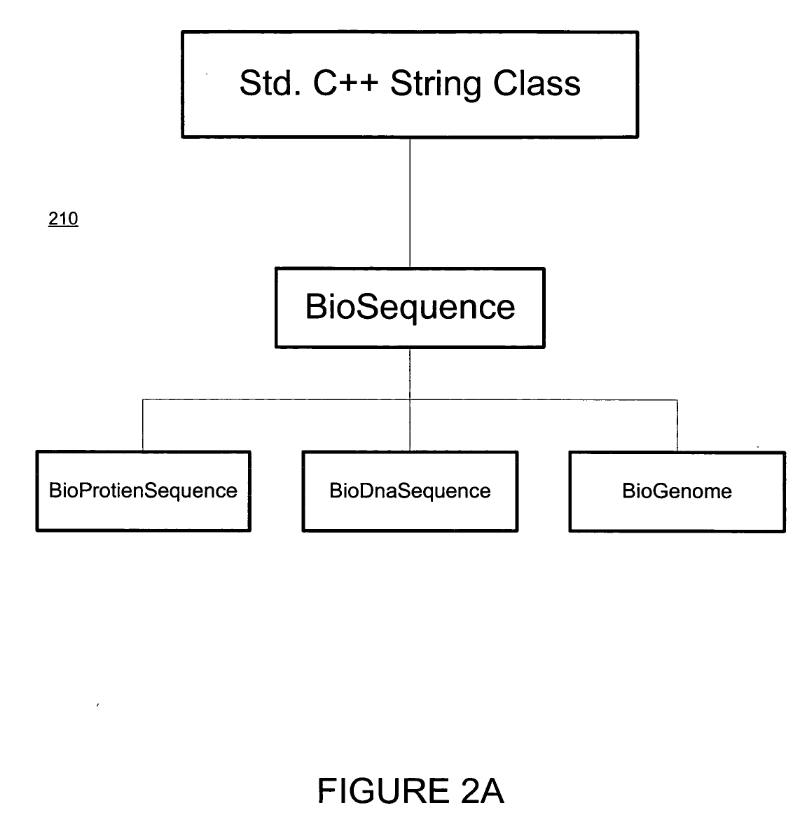

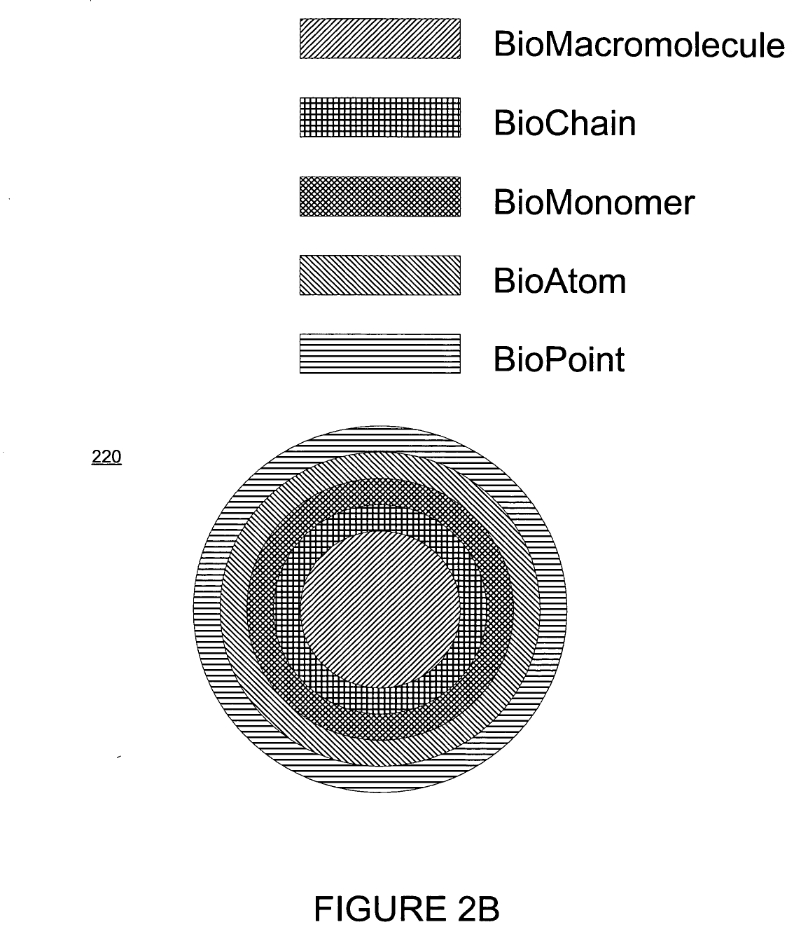

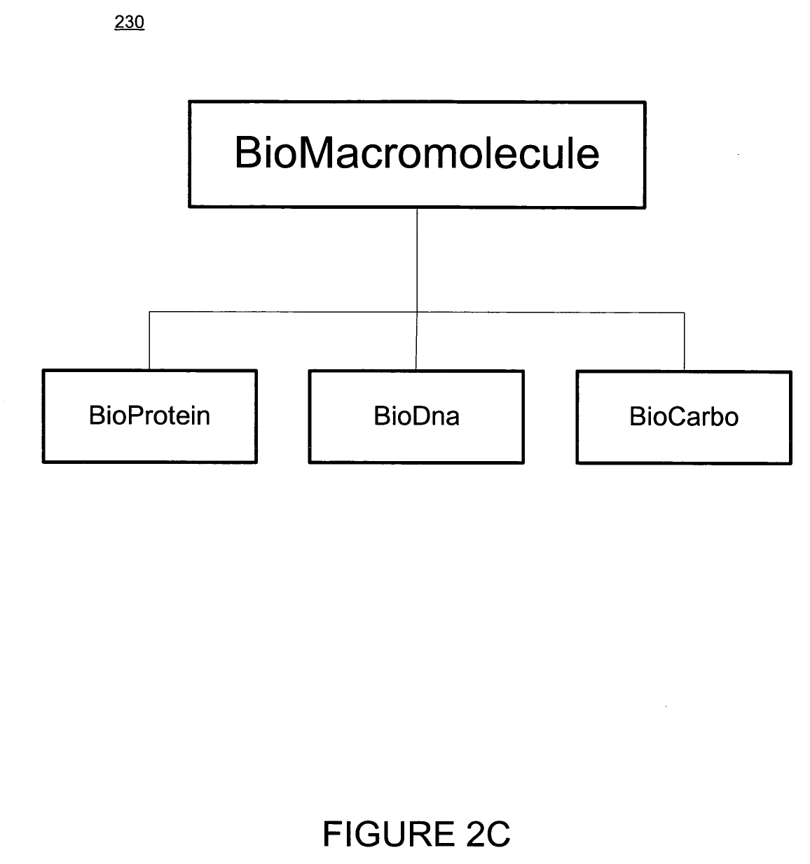

For example, multiple sequence classes may be derived from the base BioSequence class, wherein these derived sequence classes have common properties inherited to the base class from which they derive. In an embodiment, BioDnaSequence and BioProteinSequence classes may, for example, be derived to differentiate between protein sequences and nucleotide sequences within the sequence domain, or between additional method characteristics of a biomolecule, for example. Such a hierarchy, wherein multiple derived bioclasses 104 inherit to a common base bioclass 200, is illustrated in FIG. 2. An exemplary specific embodiment of the inherency of a common base bioclass and multiple derived bioclasses of the present invention is illustrated in FIG. 2A. In FIG. 2A, the file format of the file has been assessed, the sequence aspects of the file have been assessed, and the sequence nature of the file may be further broken down into lower hierarchical levels, as illustrated. For example, DNA, genome, and/or protein sequences, among others, may be subservient to a master bio-sequence class, which may, in turn, subserve a standard language class, such as a C++ string class.

FIG. 2B illustrates an exemplary embodiment of a derived hierarcy of the present invention. The present invention, by basing the coding in objects, may build continually outward from the basic levels of biological building blocks, as illustrated. In other words, a BioPoint may form the basis for a BioAtom, one or more BioAtoms the basis for a BioMonomer, and so on, and this inheritance may be implemented in the hierarchy of biological abstraction in the present invention.

Referring now to FIG. 2B, Biopoint may be, for example, the coordinates of an atom. For example x, y, and z coordinates together may form a biopiont. Most molecules (for example, DNA, proteins), in living organisms, contain six different atoms; hydrogen, carbon, nitrogen, phosphorous, oxygen and sulfur. It should be noted, however, that some molecules may contain atoms other than those specifically exemplified herein. These may be referred to herein as BioAtoms. Hence, BioPoint refers to x, y, z coordinates of BioAtoms. Further, it is known in the art that atoms in a molecule may be held together in a fixed orientation by, for example, covalent chemical bonds. BioMonomer herein refers to such molecules, or groups of Bioatoms, held together chiefly by covalent bonding, such as, for example, glucose, methionine, lysine, etc. A Biochain herein refers to two or more monomers held together by covalent chemical bonds. For example, amino acids are monomeric building blocks of a polypeptide chain. Likewise, monosaccharides are building blocks of polysaccharides. Biomacromolecule may refer to large biological molecules. For example, it is known that a number of interactions that are weaker than covalent bonds may help to determine the shape of many large biological molecules and to stabilize complexes of two or more different molecules. Some non-limiting examples of BioMacromolecules include glycosilated proteins, multimeric proteins or macromoleuclar assemblies. For example, it is known that multimeric proteins contain several protein subunits held together by noncovalent bonds. The protein macromolecular structures may combine with other cell biopolymers, like lipids, carbohydrates and nucleic acids, to form complex cell organelles, for example.

An additional exemplary specific embodiment of a common base bioclass, and multiple derived bioclasses derived therefrom, similar to the embodiment of FIG. 2A, and for certain of the elements illustrated in FIG. 2B, particularly the BioMacromolecule hierarchical level, is illustrated in FIG. 2C.

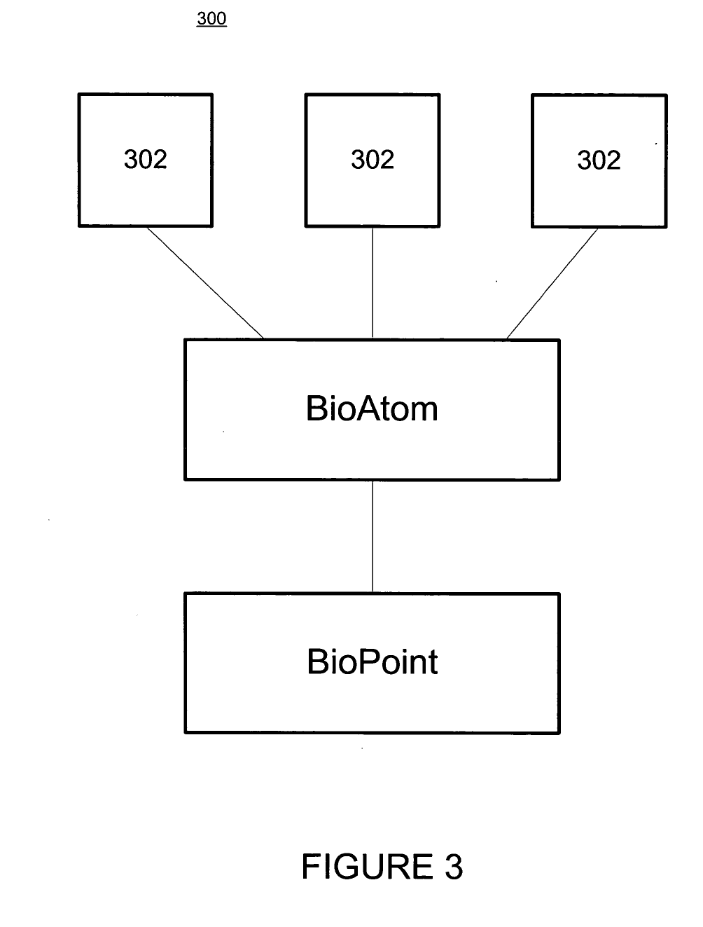

A fundamental entity of biostructure information 300 may be represented by a set of three coordinates 302, as illustrated in FIG. 3. This fundamental entity may allow for the creation of a BioPoint class in the present invention. Further, a point qualified with a name and number may become the primary entity of a chemical molecule, an atom. Thereby, a BioAtom ADT class may be created, which inherits BioPoint, as will be apparent to those skilled in the art, and as shown in FIG. 2B.

Similarly, any biomacromolecule may be defined as a polymer of a defined set of monomers. A monomer ‘contains’ a group of atoms. For example, proteins are all from a set of 20 amino acid residues. Similarly, all DNA/RNA molecules are made from a set of 5 nucleotides, namely A, C, G, T or U. Likewise, carbohydrates are formed from monosaccharides.

The difference in the number of atoms of the monomeric units of biomolecules, i.e. the different numbers of atoms in proteins, nucleic acids, or carbohydrates, for example, makes static memory allocation to store the classes correspondent thereto significantly less efficient due, in part, to differences in the required storage capacity. In order to facilitate increased storage efficiency and improved usage of memory, dynamic memory allocation (DMA), such as that available using C++ standard template libraries, is employed. Standard template libraries provide a set of sequence and association containers, such as list, vector, deque, stack, map, set, and multiset, for example. The content of each of these containers may be randomly and quickly accessed by any of numerous available methods.

A BioResidue ADT may be created to dynamically store information regarding a residue, its name, the atom information related thereto, and its number, for example, as a given file, such as a PDB file. This BioResidue ADT may be a BioResidue Class declared with a residue name, a residue number and a group of atoms, for example. The information of different atoms in the residue may then be dynamically stored using a vector container, as discussed hereinabove, for example. Similarly, BioNucleotide and BioMonosaccharide Classes may be declared, for example.

A protein, for example, may be abstracted into group of chains, with each chain having a correspondent group of residues. Thereby, similarly to the BioResidue ADT, a BioChain Class may be implemented having a vector standard template library to dynamically hold the BioResidues of the BioChain. Hence, the BioChain would be a group of BioResidues qualified with a Chain identifier. Further, a BioProtein Class may contain a group of BioChains, and a BioWater, for example, wherein the BioWater class may specially hold information about water molecules. As set forth hereinabove, structural information of each relevant bioclass may thusly be abstracted to a series of classes that are aggregated, contained or inherited from one another, independently and in accordance with biological structure behaviors.

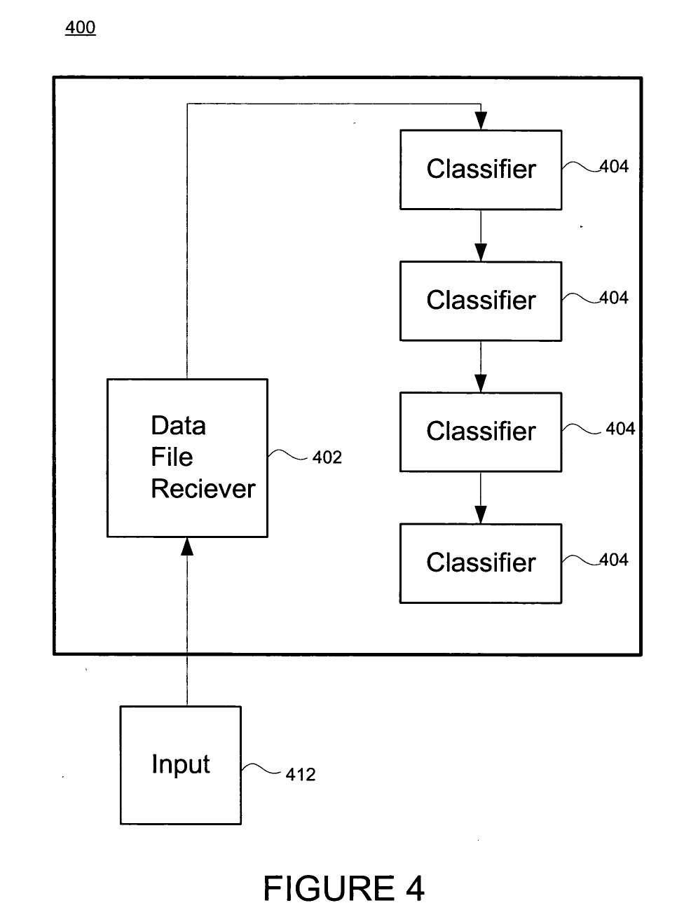

FIG. 4 is a block diagram illustrating a biological data manipulator, manipulation system, and programming hierarchy and system 400. The system of FIG. 4 includes a hierarchical organization for biodata, including at least a data file receiver 402, and first 404, second 406, and third classifier 408, wherein the first, second and third classifiers may organize data received by the data file receiver into a class and object multiple inheritance hierarchy, such as that of an object-oriented programming.

The data file receiver receives a first data file. The first data file includes data indicative of a first data file type and at least one biological data object. As used herein, a data file type, or file type, may include, for example, one or more of a plurality of file formats or languages, such as Microsoft Word, Excel, C++, Java, or the like, for example, and a data class may include classes and/or objects used in object-oriented programming, as will be known to those of ordinary skill in the art. The data file receiver that receives the first data file may be a data receiver known to those skilled in the art for receiving data, such as a hardware or software data processor, a hardware or software data memory, or a software database, for example.

The first classifier applies a plurality of rules to the first data file. These rules assess a data file type and/or a file type of the first data file. This assessing may be performed by parsing the first data file into a first data file type and into at least one class, such as a string class. The at least one class may be formed as a programming object of a predetermined class, having predetermined methods and characteristics associated therewith. The string class may be selected in accordance with the assessed data file type, for example, such as wherein the data file type is a C++ biosequence and the string class are determined accordingly.

The second classifier differentiates a master class for ones of the plurality of string classes. The master class is differentiated against a plurality of available master classes until a matching master class is obtained. The selection of master classes includes at least a single biosequence master class and a multiple biosequence master class. The single sequence master class may be hereinafter referred to as BioSequence, and the multiple sequence master class may be hereinafter referred to as BioMultipleSequence. The single sequence master class may be matched by the second classifier for reading single sequence biodata, and the multiple sequence master class may be matched by the second classifier for reading multiple sequence biodata. The multiple biosequence master class may be a grouping of single biosequence master classes. The selected master class may form a base class for derived sequence classes, such as those classified by the third or a subsequent classifier, as discussed hereinbelow. Additionally, the second classifier may be scalable by addition of ones of the master classes.

Further, a plurality of methods, both internal and external to the programming of the biodata manipulation system, may be applicable to the matching master class. The external methods may include, for example, external software applications and programs. The methods applicable to the selected master class may allow for manipulation of the biodata corresponding to the selected master class, in accordance with the characteristics of the selected master class. The allowed manipulations may be received as instructions from a user of the biodata manipulation system.

The third classifier classifies a biological data object of the first data file. In an embodiment, the biological data object may be multiple inherited to the master class in accordance with the rules applicable to the biological data object according to the first classifier, as will be known to those skilled in the art of object oriented programming. This multiple inheritance may occur in that all third classifier biological data objects having a first file type inherited to a second classifier master class representing that first file type. Further, this multiple inheritance may be in accordance with a partial sequence of stored biodata, such as biodata stored in a processor or memory or database associated with the biodata manipulation system, compared by the third classifier against a sequence of one of the string classes. The third classifier may be, for example, a software comparator. The stored sequence of biodata, which may be, for example, a DNA sequence, a genome, a gene, a cDNA sequence, an RNA sequence, an mRNA sequence, a tRNA sequence, a plasmid, an EST, an SNP, or an amino acid, may be compared by the comparator against each sequence of one of the string classes. The comparator may differentiate, for example, between a protein class and a nucleotide sequence class. For example, the comparator may access a codon library. The comparator may however also access any other biological data library, without any restriction. The comparator may then compare, over an entire one of the string classes, codons within the codon library (or any other biological data within a biological data library) to the sequence of the string classes until a codon match (or biological data match is obtained). This software comparator may be, for example, a software for-loop that iterates, three characters in the string class at a time, over the entire one of the string class.

Further, a plurality of methods, such as method objects, both internal and external to the programming of the biodata manipulation system, may be applicable to the biological data object. The external method objects may include, for example, external software applications and programs. The methods applicable to the selected biological data object may allow for manipulation of the biodata corresponding to the selected biological data object, in accordance with the characteristics of the selected biological data object. The allowed manipulations may be received as instructions from a user of the biodata manipulation system. The third classifier may be scalable by addition of ones of the biological data objects to select from, or by the addition of method objects to operate on the biological data objects.

In an exemplary embodiment, a manipulation available for the selected biodata class or object may be a calculation of molecular weight of the biodata object. The calculation of molecular weight may, for example, include an association of a molecular counter number with each sequence of stored biodata. Upon a match by the third classifier, an addition of the molecular counter number of the stored biodata match for the selected biological data object by the third classifier to a previous molecular total weight of previous matches, and a subtraction of a molecular weight of a water molecule, may be performed by the third classifier.

In an embodiment of a manipulation, the third classifier, or an additional classifier, such as a fourth classifier 410 multiple inherited to the second classifier, may include an amino acid library, wherein, upon location of a codon match or a biological data match by the third classifier, the third or subsequent classifier may compare the codon match against the amino acid library to obtain an amino acid match. The third, or subsequent, classifier may return a single letter code indicative of the amino acid match. Thus, over a series of iterations by the third or subsequent classifier, each returned single letter code is appended to a translated sequence string. A protein secondary structure may then be predicted from the translated sequence by a comparison on the translated sequence to at least one amino acid propensity by., for example, an external application software object. Each single letter code may additionally have associated therewith a molecular weight, a molecular volume, a surface accessibility, a secondary structure propensity, a number of atoms, and hydrophobicity index, to allow for additional manipulations.

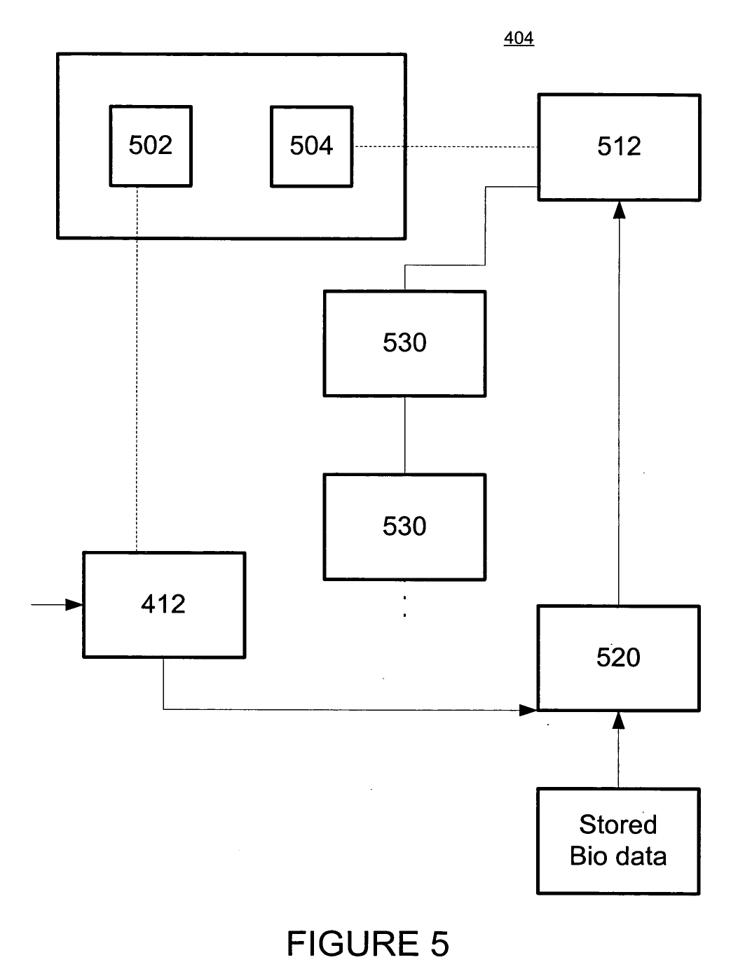

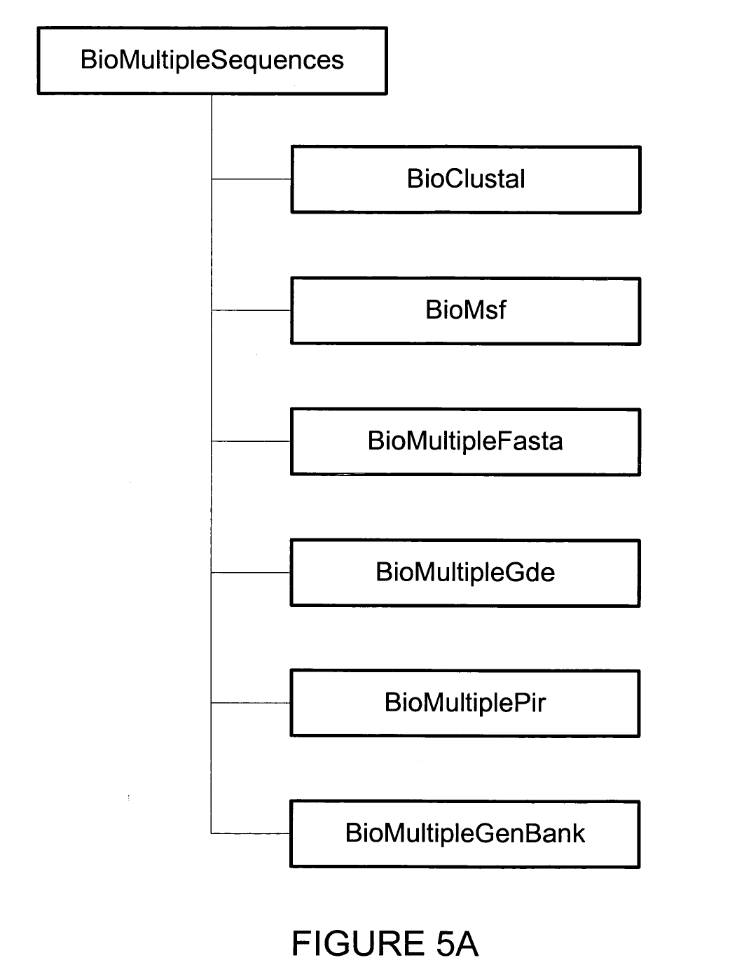

FIG. 5 is a block diagram illustrating an embodiment of a first classifier, also referred to as an abstractor 504, for use in the system of FIG. 1 of the present invention. The abstractor 504 of the present invention may include, for example, a coder 502 linked directly or indirectly to an input device 412 and initial source code 504, or the like, for example, connected to a parser 512 that is preferably used for efficient data curing, data mining, and data organization.

The parser 512 may indirectly access, such as through multiple inherited classifiers, stored biodata, or incoming data from an input, such as foreign records 520. The information stored in the foreign records 520 may be in the form of flat files and may contain information about macromolecules, which information may be indicative of a biological data class. The flat files may contain not only sequence or structure information, but also additional information such as literature references, information about function of sequences, coding regions, positions of important mutations, crystallographic information, and secondary structure information, for example. The information in the foreign records 520 may be secured in an illustrative embodiment.

The file information of the flat files may be organized into fields, each with an identifier called a record, illustratively shown herein as the first text on each line. The names and length of records may differ from one file format to another. For example, SWISSPROT and EMBL may have records of size two characters, and PDB may have a record size of maximum six characters. Each file format has correspondent thereto standardized rules, such as rules regarding format and grammar of the particular file. These rules may available at respective home pages.

Each flat file record may have associated therewith data, and may have a set of predefined properties. For example, the CRYST1 record in a PDB file contains information pertaining to unit cell and space group parameters, and may occur only once per file. This association of data and properties qualifies a record as an ADT, and hence each record described in different file formats is implemented as a Class, following the rules 512. Thereby, the abstractor 504, via the parser and multiple inherited classes associated therewith as multiple inherited classifiers, allows a user and/or developer to access and manipulate only information of interest by dividing files into smaller and simpler classes through the OO class generation process 530. In this process 530, the classes representing one file format may be multiple inherited to a master class representing the file itself. For example:

class BioGenBank : public BioGenBankLocus, public
BioGenBankDefinition, public BioGenBankVersion, public
BioGenBankAccession, public BioGenBankSegment, public
BioGenBankKeywords, public BioGenBankSource, public
BioGenBankReference, public BioGenBankFeatures, public
BioGenBankBaseCount, public BioGenBankOrigin, public
BioGenBankSequence {
public:
BioGenBank( const string& ) ;
};

Similarly, BioSwissProt and BioEmbl may share common records. Thus, to create BioSwissProt class, common records and the SWISSPROT specific classes are inherited 216 in the same manner as BioEmbl uses to create the BioEmbl class, thus allowing for code and record re-use throughout the system between related classes. The use of multiple inheritance 216 thus allows code and records to be reused efficiently. Likewise, since Fasta format is the simplest and most widely used file format, a BioFasta class may be derived from a master class, such as the BioSequence class, to read a flat file in Fasta format. Derived classes may be written in a selected database form 218 to, for example, a data storage device 150.

In an embodiment of the invention, the single sequence formats discussed hereinabove may be combined to form multiple sequence formats. Multiple sequence formats may include clustal format, multiple fasta, msf, multiplegde and multiplepir, for example. To enable the reading of multiple sequence formats by the parser 512 and the classifiers multiple inherited therefrom, a base class called BioMultipleSequence may be created, such as by the input 412 or the initial source code 504. BioMultipleSequence preferably contains a group of BioSequences generated by the OO class generator 530.

The BioMultipleSequence class may be an STL container, and may be a map association container containing a key and an associated value. Thereby, this class may be accessed from a data storage device, for example, using a value through a key. The key and value may be valid datatypes or user defined data structures. For example, in the BioMultipleSequence class, an int and Biosequence may be associated.

Inter-multiple sequence format converters may be incorporated as methods into the BioMultipleSequence class. Thus, by creating BioMultipleSequence base class, programs such as BOXSHADE and CLUSTALW may be added as methods. BioClustal, BioMsf, BioMultipleFasta, BioMultipleGde, BioMultiplePir, for example, may be classes derived from BioMultipleSequences which read respective file formats. As these derived multiple sequence classes are derived from BioMultipleSequence Class, which represents combined ones of the BioSequence class, irrespective of the format in which the files are read, the user may convert received records into any desired format from within any derived multiple sequence class, thereby allowing a multiple sequence of interest to be operated using the operations provided in the BioSequence class. An exemplary embodiment of the derived BioMultipleSequences class is illustrated in FIG. 5A.

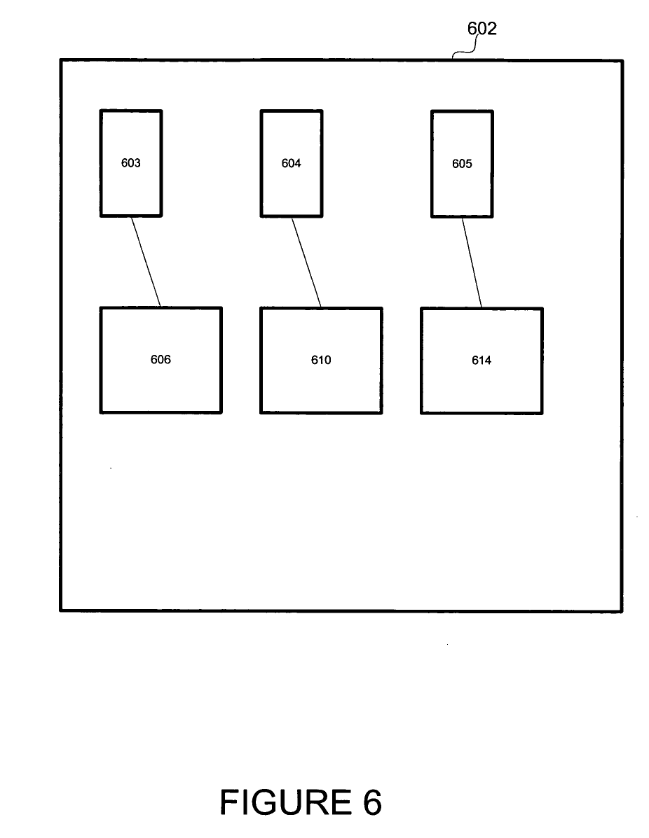

FIG. 6 is a block diagram illustrating an embodiment of a data library 602 for use in the present invention. A data library 602 of the present invention may include, for example, an initializer 604, a BioAminoAcid Library 606, a BioNucleicAcid Library 610, and a BioAtom Library 614. Each library in the data library 602 allows access to properties characterized by a set of attributes. For example, in a BioAminoAcid Library 606, every amino acid has a respective molecular weight, molecular volume, surface accessibility, Chou & Fasman Secondary structure propensity, number of atoms, and hydrophobicity index. Likewise, for example, in a BioNucleicAcid Library 610, every nucleic acid has a single letter nucleotide codes, nucleic acid name, molecular weight, complementary base, an RNA base and so on.

In an embodiment, each library in the data library 602 may be initialized by its own initializer (603, 604, 605) before accessing parameters associated with the respective libraries.

For example, code correspondent to the data libraries may include:

string BioDnaSequence: :getTranslatedSequence( ) {
string y;
BioNucleicAcidLibrary::codonInit ( );
BioArninoAcidLibrary::initialised ( );
for ( int i =0; i< sequence_.length() ;i+=3) {
y+=BioAminoAcidLibrary::AminoAcid (BioNucleicAcidLibrary::
StdCodonTable[ upperCase( sequence_.sub str(i,3 ) ) ]
.getSingleLetterCode( ); }
return y; }

In addition to the well known standard codon table, other codon tables containing unique codons associated with a set of cell organelles (e.g., CAG as a start codon in the codon table for mitochondria) or a given set of organisms (e.g., codons for Valine as a start codon in the codon table for bacteria, Pseudomonas sp., Staphylococcus sp.) may also be provided as part of the data library. As stated earlier, beside a codon library any other biological data library can be used, the term “codon library” in this application has therefore to be understood both in the sense of a direct codon library, but also in the sense of a biological data library in a more general sense. Also the term “codon” as used in this application should also cover biological data in a more general sense.

Before accessing data from the data libraries, a respective library, for example BioAminoAcidLibrary, may be to be initialized with a static member function, such as, for example, CodonInito ( ) to access the codon table. Similarly, when initialised ( ) function is activated, for example, the amino acid information and attributes may be accessed from BioAminoAcidLibrary. Additionally, Sequence_.length( ) may give the total length of the sequence stored after reading an annotated file such as, for example, GenBank.

Thus, through an iterating for-loop, for example, a sequence may be iterated in or against a library sequence a predetermined number of characters, such as three characters, at a time. For example, by using sequence_.substr (i, 3), a three letter sub-string is held. This three letter string may be passed to BioNucleicAcidLibrary::StdCodonTable[upperCase(sequence_.b). Using the stored three letter string, BioNucleicAcidLibrary::StdCodonTable may return the amino acid corresponding to that three letter string. This amino acid may be passed to BioAminoAcidLibrary::AminoAcid[ ] as an argument. To obtain a single letter code for the amino acid passed as argument, method ‘getSingleLetterCode( )’ may be accessed, which method returns the single letter code of that AminoAcid from the StdCodonTable. This returned single letter code may be continuously appended to a string y which is returned to method ‘getTranslatedSequence’ to obtain the complete, translated amino acid sequence, i.e. the protein.

Similarly, the molecular weight of a protein sequence may be calculated. For example, an embodiment of the code include:

double BioProteinSequence: :getMolecularWeight( )
{
double mw;
map<string, BioAminoAcidLibrary> : : const_iterator ci;
string::const_iterator si;
BioAminoAcidLibrary: :initialised( );
tor( si = sequence_.begin( );si != sequence_.end( ); si++) {
for (ci = BioAminoAcidLibrary::AminoAcid.begin(); ci !=
BioAminoAcidLibrary: :AminoAcid.end() ;ci++ ) {
if( (*si) == (*ci).second.getSingleLetterCode( ) )
mw += (*ci).second.getMolecularWeight ( )− 18.00; }
}
return mw; }

In this example of calculation of the molecular weight of a given protein sequence, two constant iterators traverse the AminoAcid Container and the query sequence of which the molecular weight is to be calculated. When the character of the query sequence is identical to the single letter code in the AminoAcid container, the counter number of molecular weight of that amino acid is added continuously, and the molecular weight of a water molecule is subtracted continuously, to iteratively obtain the molecular weight. The total summation over all characters in the query sequence yields the molecular weight of the protein sequence.

Similarly, using the data libraries, protein secondary structure may be predicted from the query sequence, due to the fact that the BioAminoAcidLibrary provides properties, such as Chou & Fasman propensities, for example, for each amino acid. To access the atomic mass of carbon atom from the BioAtomLibrary, the following code may be utilized:
BioAtomLibrary : : initialised( ),.
cout<<Element[“C”].getAtomicMass( )<<endl;

Further, the hierarchical class organization of the present invention allows simplistic communication between domains. For example, a sequence from an Embl database and CDS may be translated and then aligned with a sequence given in the Atom record, not using Seqres. Exemplary code to perform this might include:
BioEmbl hy (‘p53.embl’};
BioChain hyp2 (‘p52.pdh’};
BioAlign aln ( hy.getTranslatedSequence (1234, 1788), hyp2.getSequence( )),.

Further, to keep the number of functions and/or methods to be memorized by a researcher in the present invention to a minimum, the constructors and/or the methods may be overloaded. For example:
a) BioChain( );
is a constructor that may be used to instantiate an empty chain and then later populate it with relevant information using pushXXX methods;
b) BioChain ( const string& );
is a constructor used wherein the PDB file name is given as the argument. It reads the first chain and stops from reading later chains. The chain termination may be through TER, BREAK or END records or OXT string names, for example;
c) BioChain (const string&, char );
allows a chain to be loaded by giving the PDB file name as first argument and giving the desired chainID as the second argument;
d) BioChain ( char chid; vector<BioResidue>);
is a constructor that allows a group of residues held together in a vector STL to be converted as a BioChain datastructure. This method of converting may be employed, for example, to allow for use of the methods provided in BioChain Class; e) BioChain (long atnumber,string atname,string resname, char ch, longresnumber, double xI, doubleyI, double zI, double ocI, double bfI, string atrec); allows other constructors to read the information in different ways, and finally populate the BioChain using this constructor.

The following example projects function overloading:

GetMean( ), BioMatrix, getHelixDirectionCosines( ) method
BioMatrix BioPdbHelix: :getHelixDirectionCosines( const
string& file) {
BioChain ss = getHelixCoordinates(file); return
getHelixDirectionCosines( ss ); }
BioMatrix BioPdbHelix: : getHelixDirectionCosines(BioChain&
ss ) {
vector<double>l1;
vector<double>m1 ; vector<double>n1 ;
BioMatrix lmn(3, 1 );
if ( ss.getNumberOfR.esidues( ) >= 4) {
for ( int i =1; i< ss.getNurnberOfR.esidues( ) −2; i++)
{
BioAtom c1 = ss.getResidue(i−1).getAtom(“CA”);
BioAtom c2 = ss.getResidue(i).getAtom(“CA”);
BicAtom c3 = ss.getResidue(i+ l).getAtom(“CA”);
BioAtom c4 = ss.getResidue(i+2).getAtom(“CA”);
double 1=O.O;double m=O.O;double n=O.O;
helixAxisDirectionCosines( c l,c2,c3 ,c4,l,m,n);
l1.push_back(l);
m1.push_back(m); n1.push_back(n); }
lmn[O][0]=BioStatistics : :getMean(ll );
lmn[1 ][0]=BioStatistics: :getMean(ml );
lmn[2][0]=BioStatistics: :getMean(nl ); }
return lmn; }

In an exemplary embodiment of the present invention, a macromolecular crystallographic class, herein referred to as BioHKL class, may be created to, for example, read Denzo processed h, k, I and intensity files. This class may incorporate, as member functions, crystallographic programs, such as those for finding intensity statistics, computing intensive refinement algorithms, or solving structures, for example.

A BioAlign class may contain algorithms for sequence alignment, such as I Local Alignment, Global Alignment, and n-tuple Algorithms used in Blast and Fasta, for example. Each algorithmic method class may be accessible to other classes having properties that make accessibility to that algorithmic method class practicable.

A file parser class may also be preferably included in the present invention. All file parsers for the classes of the biodata management system may be included in this class. The file parser class may read a line of flat file data and stores that line as a C++ string class. This class may include static functions, such as readstring( ), readDouble, readLong( ), which may return string, double or long values, respectively, dependently upon the starting and ending positions given as arguments to the static function. Thereby, the rules and grammar of different file formats are implemented by this class to extract desired information. For example, the following implementation of BioProtein illustrates the extraction of atom/residue information is extracted from an ATOM record, using a file parser class called BioHelperClass, from a PDB file:
String at_Name=BioHelperClass::readString (line2.12, 15);
long at_Number=BioHelperClass::readLong (line2.6.10);
string resName=BioHelperClass::readString (line2, 17, 19);
long resNumber=BioHelperClass::readLong (line2.22.25);
double x-=BioHelperClass::readDouble (line2.30, 37);
double y-=BioHelperClass::readDouble (line2, 38.45);
double z-=BioHelperClass::readDouble (line2.46, 53);
double oc-=BioHelperClass::readDouble (line2, 54, 59);
double bf-=BioHelperClass::readDouble (line2.60.65);
string at_Record-=line2.substr (0, 6);
char chid=line2[21];

A BioMatrix class may additionally be included in the present invention. BioMatrix may be a class designed to perform matrix manipulations, such as matrix multiplication, thereby creating dynamic arrays. In an exemplary embodiment, the, *, operator has been overloaded, which may simplify coding as will be apparent to those skilled in the art.

A BioStatistics class may be used to calculate mean, maximum, minimum, standard deviation, variance and/or other statistical utilities of a given data set. These methods are static. The data may be passed to the static method as contained in a vector STL. It will be apparent to those skilled in the art that other statistical descriptors may be added in, or in addition to, this class, such as basic utility functions including BioDistance( ), BioAngle( ), BioTorsion( ), BioDirectionCosines( ), BioDifference Vector( ), Bio VectorCrossProduct( ), BioDotProduct( ), BioNormalize( ), BioDotMagnitude( ), toDegrees( ), toRadians( ), uppercase ( ), lowerCase ( ), rmBlank( ), and the like. These utility functions may be coded into a BioUtilities header file.

Numerous other classes and libraries may be included in the present invention, such as, but not limited to, a BioScoringMatrixLibrary, which might include Blossum62, PAM250 and other substitution matrices, a BioSpaceGroupLibrary, an Exception and Error Handling Library, a visualization class, a

vector class, and/or a URL class. Further, the DataLibrary may be provided with information on geometrical parameters like standard bond angles, bond distances and torsion angles.

In a specific illustrative embodiment of the present invention, the manipulation and management system may include 80 Classes with approximately 100 methods in total. Each class may have a signature string prefixed “Bio”, continued with the relevant entity name, such as BioProtein, BioGenBank, BioPdbSeqres, and BioEmblGn. Method names may start with a lower case letter. For example, the first word of the name may be a descriptive verbs, such as get, show, push, or pop. The subsequent words in the name may start with an upper case letter, such as getHelixDirectionCosines( ). For example, ‘pushXXX’, such as pushResidue, pushchain, and pushAtom interface methods may be used to populate different bio-entities such as residue, chain, or atom. Non-member functions having classes as arguments may start with the “Bio” signature, and subsequent words may start with an upper case letter, such as wherein BioDistance( ) is a function that takes two BioAtoms or two BioPoints as arguments to calculate the distance, and returns the distance as a double. As shown, in a preferred embodiment, nomenclature is selected to keep the names intuitive to the researcher.

In a coding example of this illustrative nomenclature, the getXXX function returns a datatype, such as a user defined datastructure, such as BioChain, or such as a basic data type, such as double. For example:
BioProtein jxr (‘pdb2JXR.ent’);
jxr.showAllChains( );
cout<<xx.getChain (O ).getNumberOjResidues( )<<endl;
cout<<xx.getChain (1).getNumberOjResidues( )<<endl;
BioChain seg=jxr.getChainSegment (25, 85, “CA”);
   wherein “seg” is an instance of BioChain that is instantiated and assigned only the CA atoms of the residues obtained from 25th to 85th residue from pdb2JXR.ent.

In this specific illustrative example, “showXXX” function shows the results as standard output, by default, or the results may be written into a file. For example:
BioPoint x (3.4, 4.5, 5.6);
x.showPoint( );

By default, this passes ‘cout’ as the argument. In the first showPoint( ), ‘cout’ is the default value, such as the terminal or console output. In the second showpoint, the coordinates will be written to the file named “output”. This gives the researcher an opportunity to check results before storing or working on those results. In ‘show XXX’ functions, the user may thus pass the file pointer.
For example:
BioGenBank x (“genbank.txt’)
String z=x.getSequenceSegment (35, 43);
BioSequence zz (“pq55”, z);
BioEmbl g (“emblgene.txt’),.
string y=g.getSequenceSegment (103, 133);
BioSequence yy (“pr ”,y);
zz.showDotPlot (yy, “pq55.dotplot’);

In this specific illustrative example, the file “pq55.dotplot” contains the dotplot of sequences in zz and yy. Further, in this example, a BioSequence class is instantiated with a constructor. The BioSequence constructor expects a sequence name as first argument, and the corresponding sequence as second argument. The function showDotPlot plots the identity between two sequences in ascii format. The user may-further employ the local alignment method in BioSequence class to give a relevant match, mismatch, and gap penalty as arguments in the method.

Accordingly, by practicing one or more of the above embodiments, in combination with a compiler-interpretor, one can arrive at an object oriented biological analysis framework.

It will be apparent to those skilled in the art that the bio-platform of the present invention, and particularly as disclosed herein throughout, such as, but not limited to, with respect to FIG. 1, may be accessed locally, or remotely, such as via a computer network, such as an internet, an intranet, and extranet, or such as via, for example, a radio network, such as a cellular telephone, infrared, or RF network. The bio-platform of FIG. 1 icreases efficiency and decreases time for analyzing, developing, and/or manipulating biological concepts and modules, as such concepts and models may be readily imported and engaged by the bio-platform of the present invention, without significant need for programming or re-programming to allow for operations on a variety of data of differing types or differing formats. Access to the bio-platform or the object oriented biological analysis framework may be provided for a subscription fee or without a fee to subscribers or users. The subscribers or users to such information would include, for example, persons or businesses in the drug design, gene discovery and genomics research fields. Further, the bio-platform of the present invention may provide for development of bio-applications, web-enabled analysis, web-enabled educational programs and training courses, and such other applications are nonetheless within the bio-platform, and hence within the present invention.

It will be apparent to those skilled in the art that various modifications and variations may be made in the apparatus and method of the present invention without departing from the spirit or scope of the invention. Thus, it is intended that the present invention cover the modification and variations of this invention, provided those modifications and variations come within the scope of the claims made herein and the equivalents thereof.

1. A biological data manipulation system, comprising:
a first data file receiver for receiving a first data file comprising data indicative of a first data file type and data indicative of at least one biological data object;
a first classifier that applies a plurality of rules to the first data file to parse the first data file into a first data file type and into a plurality of string classes;
a second classifier that differentiates a master class for ones of the plurality of string classes, wherein the master class is differentiated against at least one selected from the group consisting of a single biosequence master and a multiple biosequence master;
a third classifier that classifies an at least one biological data object of the first data file, wherein the at least one biological data object is multiple inherited to the master class in accordance with at least one of the plurality of rules, and in accordance with at least a partial sequence of stored biodata compared by the third classifier against at least a partial sequence of at least one of the plurality of string classes.

2. The biological data manipulation system of claim 1, further comprising a plurality of methods applicable to the at least one biological data object.

3. The biological data manipulation system of claim 1, further comprising a plurality of methods applicable to the master class.

4. The biological data manipulation system of claim 3, wherein at least one of said plurality of methods provides for a user manipulation of the first data file.

5. The biological data manipulation system of claim 4, wherein the user manipulation includes a calculation of molecular weight.

6. The biological data manipulation system of claim 5, wherein the calculation of molecular weight comprises an association of a molecular counter number with each partial sequence of stored biodata, and, upon a match by said third classifier, an addition of the molecular counter number to a current one of the match by said third classifier to a previous molecular total number of previous ones of the matches by said third classifier, and a subtraction of a molecular weight of a water molecule.

7. The biological data manipulation system of claim 4, wherein at least one of said plurality of methods comprises an application software external to the biological data manipulator, and wherein a user request for the user manipulation calls the application software.

8. The biological data manipulation system of claim 1, wherein said third classifier comprises a comparator, and wherein the at least partial sequence of biodata comprises at least one selected from the group consisting of a DNA sequence, a genome, a gene, a cDNA sequence, an RNA sequence, an mRNA sequence, a tRNA sequence, a plasmid, an EST, an SNP, and an amino acid, and wherein the comparator compares the at least one selected from the group against the partial sequence of one of the string classes.

9. The biological data manipulation system of claim 8, wherein a partial sequence of the string class comprises a sequence of codons.

10. The biological data manipulation system of claim 1, wherein the single biosequence master class enables reading of a single biosequence file format.

11. The biological data manipulation system of claim 1, wherein the multiple biosequence master class enables reading of a multiple biosequence file format.

12. The biological data manipulation system of claim 11, wherein the multiple biosequence master class comprises a group of single biosequence master classes.

13. The biological data manipulation system of claim 1, wherein the third classifier accesses a codon library.

14. The biological data manipulation system of claim 13, wherein the third classifier compares codons within the codon library to the at least a partial sequence of the plurality of string classes until a codon match is obtained, over an entire one of the string classes.

15. The biological data manipulation system of claim 14, wherein the comparison of codons within the codon library comprises a software for-loop that iterates, three characters in the strong class at a time, over the entire one of the string class.

16. The biological data manipulation system of claim 14, further comprising a fourth classifier that comprises an amino acid library, wherein, upon location of a codon match by said third classifier, said fourth classifier compares the codon match against the amino acid library to obtain an amino acid match.

17. The biological data manipulation system of claim 16, wherein said fourth classifier returns a single letter code indicative of the amino acid match.

18. The biological data manipulation system of claim 17, wherein, over a series of iterations by said fourth classifier, each returned single letter code is appended to a translated sequence string.

19. The biological data manipulation system of claim 18, wherein a protein secondary structure is predicted from the translated sequence by a comparison on the translated sequence to at least one amino acid propensity in an external application software.

20. The biological data manipulation system of claim 17, wherein each single letter code has associated therewith at least a molecular weight, a molecular volume, a surface accessibility, a secondary structure propensity, a number of atoms, and hydrophobicity index.

21. The biological data manipulation system of claim 1, wherein the multiple inheritance comprises all third classifier biological data objects having a first file type inherited to a second classifier master class representing that first file type.

22. The biological data manipulation system of claim 1, wherein said third classifier differentiates between a protein class and a nucleotide sequence class.

23. The biological data manipulation system of claim 1, wherein said third classifier is scalable by addition of ones of the at least one biological data object.

24. The biological data manipulation system of claim 1, wherein said second classifier is scalable by addition of ones of the master classes.

25. The biological data manipulation system of claim 1, wherein said mater class comprises a base class for derived sequence classes.

26. The biological data manipulation system of claim 25, wherein the at least one biological data object comprises the derived sequence classes.

27. The biological data manipulation system of claim 1, wherein said third classifier further comprises a residue data class, wherein unclassified ones of the partial sequences of the plurality of string classes are classified by said third classifier to the residue data class.

28. The biological data manipulation system of claim 1, wherein said second classifier employs dynamic memory allocation.

29. The biological data manipulation system of claim 1, wherein the at least a partial sequence of stored biodata comprises at least one flat file formatted database.

30. The biological data manipulation system of claim 29, wherein the at least one flat file formatted database comprises at least one data item selected from the group consisting of biosequence information and biostructure information.

31. The biological data manipulation system of claim 30, wherein the at least one flat file formatted database further comprises at least one data item selected from the group consisting of literature references, sequence functions, coding regions, mutations, crystallographic information, and secondary structure information.

32. The biological data manipulation system of claim 31, wherein each of the selected data items is organized into a field, and wherein each field has an identifier.

33. A computer-readable medium carrying one or more sequences of instructions for manipulating biodata, wherein execution of the one or more sequences of instructions by one or more processors causes the one or more processors to perform the steps of:
receiving a first data file comprising data indicative of a first data file type and data indicative of at least one biological data object;
applying a plurality of rules to the first data file to parse the first data file into a first data file type and into a plurality of string classes;
differentiating a master class for ones of the plurality of string classes, wherein the master class is differentiated against at least one selected from the group consisting of a single biosequence master and a multiple biosequence master;
classifying an at least one biological data object of the first data file;
multiple inheriting the at least one biological data object to the master class in accordance with at least one of the plurality of rules, and in accordance with comparing at least a partial sequence of stored biodata against at least a partial sequence of at least one of the plurality of string classes.

34. The computer-readable medium of claim 33, further comprising applying a plurality of methods to the at least one biological data object.

35. The computer-readable medium of claim 33, further comprising applying a plurality of methods to the master class.

36. The computer-readable medium of claim 35, further comprising applying a plurality of methods to at least one of the master class and the at least one biological data object in accordance with a user manipulation request for the first data file.

37. The computer-readable medium of claim 36, wherein said applying a plurality of methods to at least one of the master class and the at least one biological data object comprises applying an external application software, and further comprising calling the external application software in accordance with the user manipulation request.

38. The computer-readable medium of claim 33, wherein the at least partial sequence of biodata comprises at least one selected from the group consisting of a DNA sequence, a genome, a gene, a cDNA sequence, an RNA sequence, an mRNA sequence, a tRNA sequence, a plasmid, an EST, an SNP, and an amino acid, and wherein said comparing at least a partial sequence of stored biodata comprises comparing the at least one selected from the group against the partial sequence of one of the string classes.

39. The computer-readable medium of claim 33, wherein a partial sequence of the string class comprises a sequence of codons.

40. The computer-readable medium of claim 33, wherein said classifying comprises accessing a codon library.

41. The computer-readable medium of claim 40, wherein said classifying comprises comparing codons within the codon library to the at least a partial sequence of the plurality of string classes, until a codon match is obtained, over an entire one of the string classes.

42. The computer-readable medium of claim 41, wherein said comparing codons within the codon library comprises iterating a for-loop, three characters in the strong class at a time, over the entire one of the string class.

43. The computer-readable medium of claim 33, wherein the stored biodata comprises a codon library, and wherein said classifying comprises comparing the codon library match to the at least a partial sequence of at least one of the plurality of string classes to an amino acid library to obtain an amino acid match.

44. The computer-readable medium of claim 43, further comprising associating with each amino acid match at least a molecular weight, a molecular volume, a surface accessibility, a secondary structure propensity, a number of atoms, and hydrophobicity index.

45. The computer-readable medium of claim 33, wherein said differentiating differentiates between a protein class and a nucleotide sequence class.

46. The computer-readable medium of claim 33, wherein said differentiating comprises dynamically allocating a memory associated with at least one of the one or more processors.

47. A method of providing for biodata manipulation, comprising:
receiving a first data file comprising data indicative of a first data file type and data indicative of at least one biological data object;
applying a plurality of rules to the first data file to parse the first data file into a first data file type and into a plurality of string classes;
differentiating a master class for ones of the plurality of string classes, wherein the master class is differentiated against at least one selected from the group consisting of a single biosequence master and a multiple biosequence master;
classifying an at least one biological data object of the first data file;
multiple inheriting the at least one biological data object to the master class in accordance with at least one of the plurality of rules, and in accordance with comparing at least a partial sequence of stored biodata against at least a partial sequence of at least one of the plurality of string classes.

48. The method of claim 47, further comprising applying a plurality of methods to the at least one biological data object.

49. The method of claim 47, further comprising applying a plurality of methods to the master class.

50. The method of claim 49, further comprising applying a plurality of methods to at least one of the master class and the at least one biological data object in accordance with a user manipulation request for the first data file.

51. The method of claim 50, wherein said applying a plurality of methods to at least one of the master class and the at least one biological data object comprises applying an external application software, and further comprising calling the external application software in accordance with the user manipulation request.

52. The method of claim 47, wherein the at least partial sequence of biodata comprises at least one selected from the group consisting of a DNA sequence, a genome, a gene, a cDNA sequence, an RNA sequence, an mRNA sequence, a tRNA sequence, a plasmid, an EST, an SNP, and an amino acid, and wherein said comparing at least a partial sequence of stored biodata comprises comparing the at least one selected from the group against the partial sequence of one of the string classes.

53. The method of claim 47, wherein said classifying comprises comparing codons within a codon library to the at least a partial sequence of the plurality of string classes, until a codon match is obtained, over an entire one of the string classes.

54. The method of claim 47, wherein the stored biodata comprises a codon library, and wherein said classifying comprises comparing the codon library match to the at least a partial sequence of at least one of the plurality of string classes to an amino acid library to obtain an amino acid match.

55. The method of claim 55, wherein said differentiating comprises dynamically allocating a memory.

56. A biodata programming system, comprising:
means for receiving a first data file comprising data indicative of a first data file type and data indicative of at least one biological data object;
means for applying a plurality of rules to the first data file to parse the first data file into a first data file type and into a plurality of string classes;
means for differentiating a master class for ones of the plurality of string classes, wherein the master
class is differentiated against at least one selected from the group consisting of a single biosequence master and a multiple biosequence master;
means for classifying an at least one biological data object of the first data file;
means for multiple inheriting the at least one biological data object to the master class in accordance with at least one of the plurality of rules, and in accordance with a comparison of at least a partial sequence of stored biodata against at least a partial sequence of at least one of the plurality of string classes.
